(19) United States
(12) Patent Application Publication
(10) Pub. No.: US 2001/0012829 A1
(43) Pub. Date: Aug. 9, 2001

(54) Transepithelial delivery GLP-1 derivatives

(76) Inventors: Keith Anderson, San Diego, CA (US); Henrik Agerso, Fredensborg (DK)

Correspondence Address: Novo Nordisk of North America, Inc., Suite 6400, 405 Lexington Avenue, New York, NY, 10017, US

(21) Appl. No.: 09/757,788

(22) Filed: Jan. 10, 2001

Related U.S. Application Data
(60) Provisional application No. 60/177,002, filed on Jan. 19, 2000.

(30) Foreign Application Priority Data
Jan. 11, 2000 (DK) 2000 00030

(51) Int. Cl. A61K 9/12; A61K 38/17

(52) U.S. Cl. 514/12; 424/43

(57) ABSTRACT

The present invention relates to a new formulation comprising a stabilized GLP-1 compound, such as an analog, fragment or derivative thereof for delivery across pulmonary tissue in vivo.

### BACKGROUND

The physico-chemical characteristics, production and purification methods, in vitro and in vivo potencies and clinical advantages of GLP-1 and analogues have been well-characterised over recent years. There is little doubt that the development of a pharmaceutically useful form of GLP-1, or an analogue thereof, would result in a valuable addition to the available chemotherapeutic products for the treatment of diabetes and other metabolic disorders.

It has been made clear that certain fatty-acyl derivatives of GLP-1 are prone to non-covalent self-association, which can lead to clinical failure (cf. Clodfelter D K et al. Effects of non-covalent self-association on the subcutaneous absorption of a therapeutic peptide. Pharm Res 15(2) (1998) 254-262). Furthermore, recent disclosures suggest that such derivatives require co-addition of surfactants to ensure a stabilized, therapeutically useful dosage form (cf. WO 99/29336). Importantly, due to toxicity concerns, many of the surfactants provided for in (cf. WO 99/29336) will limit the use of formulations of such derivatives to the gastrointestinal, transdermal, or possibly nasal delivery routes.

### SUMMARY OF INVENTION

The present invention relates to a new formulation comprising a stabilized GLP-1 compound, such as an analog, fragment or derivative thereof for delivery across pulmonary tissue in vivo.

We have discovered that a family of fatty-acylated GLP-1 compounds can be solubilized to a very high degree in water without formation of insoluble physical aggregates (>5 mg/mL), are stable in solution without the requirement of additional stabilizing excipients in the formulation (eg. surfactants, cyclodextrins, etc.), are physically stable in solution in the presence of external stresses such as during exposure to high shear encountered during jet or ultrasonic, nebulisation and are physically stable without forming insoluble aggregates or fibrillated products over time, and are metabolicly stable. Further, the solution structure of these candidates allow for simple formulation design changes to control pulmonary absorption rates, thus having features which allow optimisation of drug delivery. Moreover, the development of a pulmonary dosage form of a GLP-1 compound whereto is attached a lipophilic substituent optionally via a spacer represents a non-invasive means of protein drug delivery without the inconvenience and health/environmental risks associated with traditional injectable, needle-based medications.

Currently there is a need to limit the use of surfactants for developing physically stable GLP-1 solutions, such as described in (WO 99/47160, WO 99129336), when developing pulmonary delivery systems for such compounds. Unfortunately, surfactants, cyclodextrins and other potential excipients utilised for stabilising peptide solutions are associated with solubilisation of lipid components of cell membranes, and therefore, are associated with cell toxicity. In parallel, many efforts have been taken to enhance the permeability of insulin across pulmonary tissue by using formulation additives (cf. Patton J S, Bukar J, Nagarajan S. Inhaled insulin. Adv Drug Del Rev 35 (1999) 235-247), however it is very difficult to delineate the permeability enhancement effects from the toxic effects of excipients on pulmonary tissue. Thus far, permeability enhancers or absorption promoters are viewed as potentially toxic agents and will require much documentation to prove that they represent no potential harm to human subjects, especially when concerning such sensitive tissues as in the lung. One class of potentially approvable enhancers are the protease inhibitors, however they are often required in excessive amounts to improve the delivery efficiency (cf. Patton J S, Bukar J, Nagarajan S. Inhaled insulin. Adv Drug Del Rev 35 (1999) 235-247).

### DESCRIPTION OF INVENTION

A simple system is used to describe the GLP-1 compounds of the present invention. For example, Gly⁸-GLP-1(7-37) designates a peptide which relates to GLP-1(1-37) by the deletion of the amino acid residues at positions 1 to 6 and the substitution of the naturally occurring amino acid residue in position 8 (Ala) with Gly. Similarly, Lys³⁴(N^{ε}-tetradecanoyl)-GLP-1(7-37) designates (GLP-1(7-37) wherein the ε-amino group of the Lys residue in position 34 has been tetradecanoylated.

Accordingly the present invention relates to a new formulation for use in a pulmonary device, comprising a soluble and, solution stabilized, metabolic stabilized, and/or stress stabilized GLP-1 compound for delivery across pulmonary tissue in vivo.

Also, the present invention relates to a method for preparing a formulation for use in a pulmonary device, said formulation comprising a soluble and, solution stabilized, metabolic stabilized, and/or stress stabilized GLP-1 compound for delivery across pulmonary tissue in vivo.

Typical, soluble GLP-1 compounds are compounds that, within the pH range of 4-9, within a temperature range of 0-50° C., in isotonic buffered solutions, at concentrations in solution representing no less than a 1:1 potency:bioavailable dose ratio when compared to native GLP-1, demonstrate no formation of insoluble aggregates (or insoluble material), according to techniques acceptable in the art.

Typical, solution stabilized GLP-1 compounds are compounds, which, in the presence of no stabilizing excipients (e.g. surfactants, cyclodextrins, etc.), within the pH range of 4-9, within a temperature range of 0-50° C., in isotonic buffered solutions, after storage of at least 3 months, demonstrate the presence of:

No less than 90% of original protein purity, and

no formation of insoluble aggregates,

as measured by techniques acceptable in the art.

Typical, metabolic stabilized GLP-1 compounds are compounds, which demonstrate, after introduction into mammals within the therapeutic window, terminal plasma half-lives of greater than 1 hour, as measured by techniques acceptable in the art.

Typical, stress stabilized GLP-1 compounds are compounds which maintain >75% of initial bioactivity after exposure to conditions associated with manufacturing processes, delivery processes, handling or storage conditions, as measured by techniques acceptable in the art.

The therapeutic window is a range of drug concentrations within which the probability of the desired clinical response is relatively high and the probability of unacceptable toxicity is relatively low. Evans, W E ed., Applied Pharmacokinetics: Principles of Therapeutic Drug Monitoring, 3^{rd} ed, Ch 1-3, 1992.

We have discovered that a way of preparing a soluble and, solution stabilized, metabolic stabilized, and/or stress stabilized GLP-1 compound ideal for delivery across pulmonary tissue in vivo is by modifying the structure of a GLP-1 analogue by introducing a lipophilic substituent optionally via a spacer. Preferably, a spacer is present.

In one aspect the present invention relates to a pulmonary liquid or dry formulation comprising a GLP-1 compound whereto is attached a lipophilic substituent optionally via a spacer.

In another aspect the present invention relates to a method for preparing a pulmonary liquid or dry formulation for use in a pulmonary device, said formulation comprising a GLP-1 compound whereto is attached a lipophilic substituent optionally via a spacer.

In a further aspect the present invention relates to a pulmonary delivery device comprising a GLP-1 compound whereto is attached a lipophilic substituent optionally via a spacer.

In a further aspect the present invention relates to a method for preparing a pulmonary delivery device, said device comprising a GLP-1 compound whereto is attached a lipophilic substituent optionally via a spacer.

In a further aspect the present invention relates to a method of reducing blood glucose levels, treating diabetes type I, diabetes type II or obesity, or inhibiting gastric acid secretion, or inhibiting apoptosis of β-cells, comprising administering to a patient in need thereof an effective amount of a GLP-1 compound whereto is attached a lipophilic substituent optionally via a spacer, by inhalation so as to deposit said GLP-1 compound whereto is attached a lipophilic substituent optionally via a spacer in the lungs of the patient.

In a further aspect the present invention relates to a method of treating gastric ulcers comprising administering to a patient in need thereof an effective amount of a GLP-1 compound whereto is attached a lipophilic substituent optionally via a spacer, by inhalation so as to deposit said GLP-1 compound whereto is attached a lipophilic substituent optionally via a spacer in the lungs of the patient.

In a further aspect the present invention relates to use of a GLP-1 compound whereto is attached a lipophilic substituent optionally via a spacer for the preparation of a pulmonary liquid or dry formulation for reducing blood glucose levels.

In a further aspect the present invention relates to use of a GLP-1 compound whereto is attached a lipophilic substituent optionally via a spacer for the preparation of a pulmonary liquid or dry formulation for treating diabetes type I.

In a further aspect the present invention relates to use of a GLP-1 compound whereto is attached a lipophilic substituent optionally via a spacer for the preparation of a pulmonary liquid or dry formulation for treating diabetes type II.

In a further aspect the present invention relates to use of a GLP-1 compound whereto is attached a lipophilic substituent optionally via a spacer for the preparation of a pulmonary liquid or dry formulation for treating obesity.

In a further aspect the present invention relates to use of a GLP-1 compound whereto is attached a lipophilic substituent optionally via a spacer for the preparation of a pulmonary liquid or dry formulation for treating gastric ulcers.

In a further aspect the present invention relates to use of a GLP-1 compound whereto is attached a lipophilic substituent optionally via a spacer for the preparation of a pulmonary liquid or dry formulation for inhibition of apoptosis of β-cells.

In a further aspect the present invention relates to use of a GLP-1 compound whereto is attached a lipophilic substituent optionally via a spacer for the preparation of a pulmonary delivery device for reducing blood glucose levels.

In a further aspect the present invention relates to use of a GLP-1 compound whereto is attached a lipophilic substituent optionally via a spacer for the preparation of a pulmonary delivery device for treating diabetes type I.

In a further aspect the present invention relates to use of a GLP-1 compound whereto is attached a lipophilic substituent optionally via a spacer for the preparation of a pulmonary delivery device for treating diabetes type II.

In a further aspect the present invention relates to use of a GLP-1 compound whereto is attached a lipophilic substituent optionally via a spacer for the preparation of a pulmonary delivery device for treating obesity.

In a further aspect the present invention relates to use of a GLP-1 compound whereto is attached a lipophilic substituent optionally via a spacer for the preparation of a pulmonary delivery device for treating gastric ulcers.

In a further aspect the present invention relates to use of a GLP-1 compound whereto is attached a lipophilic substituent optionally via a spacer for the preparation of a pulmonary delivery device for inhibition of apoptosis of β-cells.

The lipophilic substituent may be attached to an amino group of the GLP-1 compound by means of a carboxyl group of the lipophilic substituent which forms an amide bond with an amino group of the amino acid residue to which it is attached. Alternatively, the lipophilic substituent may be attached to said amino acid residue in such a way that an amino group of the lipophilic substituent forms an amide bond with a carboxyl group of the amino acid residue. As a further option, the lipophilic substituent may be linked to the GLP-1 compound via an ester bond. Formally, the ester can be formed either by reaction between a carboxyl group of the GLP-1 compound and a hydroxyl group of the substituent-to-be or by reaction between a hydroxyl group of the GLP-1 compound and a carboxyl group of the substituent-to-be. As a further alternative, the lipophilic substituent can be an alkyl group which is introduced into a primary amino group of the GLP-1 compound.

In a further alternative, the lipophilic substituent may be attached to the GLP-1 compound by means of a spacer in such a way that a carboxyl group of the spacer forms an amide bond with an amino group of the GLP-1 compound. A spacer must contain at least two functional groups, one to attach to a functional group of the lipophilic substituent and the other to a functional group of the parent GLP-1 peptide. Examples of suitable spacers are succinic acid, lysyl, glutamyl, asparagyl, glycyl, beta-alanyl and gamma-aminobutanoyl, or a dipeptide such as Gly-Lys, each of which constitutes an individual embodiment. When the spacer is succinic acid, one carboxyl group thereof may form an amide bond with an amino group of the amino acid residue, and the other carboxyl group thereof may form an amide bond with an amino group of the lipophilic substituent. When the spacer is lysyl, glutamyl, asparagyl, glycyl, beta-alanyl or gamma-aminobutanoyl, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the ε-amino group of Lys and the lipophilic substituent. In one preferred embodiment, such a further spacer is succinic acid which forms an amide bond with the ε-amino group of Lys and with an amino group present in the lipophilic substituent. In another preferred embodiment such a further spacer is Glu or Asp which forms an amide bond with the ε-amino group of Lys and another amide bond with a carboxyl group present in the lipophilic substituent, that is, the lipophilic substituent is a N^{ε}-acylated lysine residue. In an embodiment, the spacer is an amino acid residue except Cys or Met, or a dipeptide such as Gly-Lys. For purposes of the present invention, the phrase “a dipeptide such as Gly-Lys” means any combination of two amino acids except Cys or Met, preferably a dipeptide wherein the C-terminal amino acid residue is Lys, His or Trp, preferably Lys, and the N-terminal amino acid residue is Ala, Arg, Asp, Asn, Gly, Glu, Gln, Ile, Leu, Val, Phe, Pro, Ser, Tyr, Thr, Lys, His and Trp. Preferably, an amino group of the GLP-1 compound forms an amide bond with a carboxylic group of the amino acid residue or dipeptide spacer, and an amino group of the amino acid residue or dipeptide spacer forms an amide bond with a carboxyl group of the lipophilic substituent.

Examples of such GLP-1 compounds whereto is attached one or more lipophilic substituents optionally via a spacer have been disclosed in e.g EP 0708179, WO 98/08871, WO 99/43705, WO 99/43706, WO 99/43707, WO 99/43708, WO 99/43341, which are incorporated herein by reference. The GLP-1 compounds whereto is attached one or more lipophilic substituents optionally via a spacer are useful in treatment of diabetes mellitus (types I or II) and prevention of hyperglycaemia, as well as in treatment of obesity, or gastric ulcers, or in inhibition of apoptosis of β-cells, upon administering to a patient in need thereof an effective amount of a pulmonary formulation comprising a stabilized GLP-1 compound by inhalation so as to deposit said stabilized GLP-1 compound in the lungs of the patient.

Examples of exendin as well as analogs, derivatives, and fragments thereof to be included within the present invention are those disclosed in WO 9746584 and U.S. Pat. No. 5,424,286. U.S. Pat. No. 5,424,286 describes a method for stimulating insulin release with exendin polypeptide(s). The exendin polypeptides disclosed include HGEGTFTSDLSKQMEEEAVRLFIEWLKNGGX; wherein X=P or Y, and HX1X2GTFITSDLSKQMEEEAVRLFIEWLKNGGPSSGAPPPS; wherein X1X2=SD (exendin-3) or GE (exendin-4)). The exendin-3 and -4 and fragments are useful in treatment of diabetes mellitus (types I or 11) and prevention of hyperglycaemia. They normalise hyperglycaemia through glucose-dependent, insulin-independent and insulin-dependent mechanisms. Exendin-4 is specific for exendin receptors, i.e. it does not interact with vasoactive intestinal peptide receptors. WO 9746584 describes truncated versions of exendin peptide(s) for treating diabetes. The disclosed peptides increase secretion and biosynthesis of insulin, but reduce those of glucagon. The truncated peptides can be made more economically than full length versions.

In one embodiment of the invention the GLP-1 compound is GLP-1(7-37) or GLP-1(7-36) amide.

In a further embodiment of the invention the GLP-1 compound is exendin or an analog thereof.

In a further embodiment of the invention the GLP-1 compound whereto is attached a lipophilic substituent via a spacer is Arg³⁴Lys²⁶(N^{ε}-(γ-glutamyl(N^{α}-hexadecanoyl)))-GLP-1(7-37)-OH.

In a further embodiment of the invention the GLP-1 compound whereto is attached a lipophilic substituent via a spacer is Arg¹⁸, Leu²⁰, Gln³⁴, Lys³³ (N^{ε}-(γ-aminobutyroyl(N^{α}-hexadecanoyl))) Exendin-4-(7-45)-NH₂.

In a further embodiment of the invention the GLP-1 compound whereto is attached a lipophilic substituent via a spacer is Arg³³, Leu²⁰, Gln³⁴, Lys¹⁸ (N^{ε}-(γ-aminobutyroyl(N^{α}-hexadecanoyl))) Exendin-4-(7-45)-NH₂.

In a further embodiment of the invention the GLP-1 compound is a GLP-1 analogue.

In a further embodiment of the invention the GLP-1 analogue is selected from the Thr⁸, Met⁸, Gly⁸, and Val⁸ analogues of GLP-1(7-37) and GLP-1(7-36) amide, more preferred the Gly⁸ and Val⁸ analogues of GLP-1(7-37) and GLP-1(7-36) amide, most preferred the Val⁸ analogues of GLP-1(7-37) and GLP-1(7-36) amide.

In a further embodiment of the invention the GLP-1 analogue has the formula II:

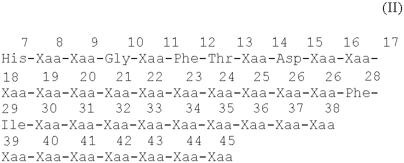

wherein

Xaa at position 8 is Ala, Gly, Ser, Thr, Leu, Ile, Val, Glu, Asp, Met, or Lys,

Xaa at position 9 is Glu, Asp, or Lys,

Xaa at position 11 is Thr, Ala, Gly, Ser, Leu, Ile, Val, Glu, Asp, or Lys,

Xaa at position 14 is Ser, Ala, Gly, Thr, Leu, Ile, Val, Glu, Asp, or Lys,

Xaa at position 16 is Val, Ala, Gly, Ser, Thr, Leu, Ile, Tyr, Glu, Asp, or Lys,

Xaa at position 17 is Ser, Ala, Gly, Thr, Leu, Ile, Val, Glu, Asp, or Lys,

Xaa at position 18 is Ser, Ala, Gly, Thr, Leu, Ile, Val, Glu, Asp, or Lys,

Xaa at position 19 is Tyr, Phe, Trp, Glu, Asp, or Lys,

Xaa at position 20 is Leu, Ala, Gly, Ser, Thr, Leu, Ile, Val, Glu, Asp, or Lys,

Xaa at position 21 is Glu, Asp, or Lys,

Xaa at position 22 is Gly, Ala, Ser, Thr, Leu, Ile, Val, Glu, Asp, or Lys,

Xaa at position 23 is Gln, Asn, Arg, Glu, Asp, or Lys,

Xaa at position 24 is Ala, Gly, Ser, Thr, Leu, Ile, Val, Arg, Glu, Asp, or Lys,

Xaa at position 25 is Ala, Gly, Ser, Thr, Leu, Ile, Val, Glu, Asp, or Lys,

Xaa at position 26 is Lys, Arg, Gln, Glu, Asp, or His,

Xaa at position 27 is Glu, Asp, or Lys,

Xaa at position 30 is Ala, Gly, Ser, Thr, Leu, Ile, Val, Glu, Asp, or Lys,

Xaa at position 31 is Trp, Phe, Tyr, Glu, Asp, or Lys,

Xaa at position 32 is Leu, Gly, Ala, Ser, Thr, Ile, Val, Glu, Asp, or Lys,

Xaa at position 33 is Val, Gly, Ala, Ser, Thr, Leu, Ile, Glu, Asp, or Lys,

Xaa at position 34 is Lys, Arg, Glu, Asp, or His,

Xaa at position 35 is Gly, Ala, Ser, Thr, Leu, Ile, Val, Glu, Asp, or Lys,

Xaa at position 36 is Arg, Lys, Glu, Asp, or His,

Xaa at position 37 is Gly, Ala, Ser, Thr, Leu, Ile, Val, Glu, Asp, or Lys, or is deleted,

Xaa at position 38 is Arg, Lys, Glu, Asp, or His, or is deleted,

Xaa at position 39 is Arg, Lys, Glu, Asp, or His, or is deleted,

Xaa at position 40 is Asp, Glu, or Lys, or is deleted,

Xaa at position 41 is Phe, Trp, Tyr, Glu, Asp, or Lys, or is deleted,

Xaa at position 42 is Pro, Lys, Glu, or Asp, or is deleted,

Xaa at position 43 is Glu, Asp, or Lys, or is deleted,

Xaa at position 44 is Glu, Asp, or Lys, or is deleted, and

Xaa at position 45 is Val, Glu, Asp, or Lys, or is deleted, or (a) a C-1-6-ester thereof, (b) amide, C-1-6-alkylamide, or C-1-6-dialkylamide thereof and/or (c) a pharmaceutically acceptable salt thereof, provided that

(i) when the amino acid at position 37, 38, 39, 40, 41, 42, 43 or 44 is deleted, then each amino acid downstream of the amino acid is also deleted.

In a further embodiment of the GLP-1 analogue of formula II, the amino acids at positions 37-45 are absent.

In another embodiment of the GLP-1 analogue of formula II, the amino acids at positions 38-45 are absent.

In another embodiment of the GLP-1 analogue of formula II, the amino acids at positions 39-45 are absent.

In another embodiment of the GLP-1 analogue of formula II, Xaa at position 8 is Ala, Gly, Ser, Thr, Met, or Val.

In another embodiment of the GLP-1 analogue of formula II, Xaa at position 8 is Gly, Thr, Met, or Val.

In another embodiment of the GLP-1 analogue of formula II, Xaa at position 8 is Val.

In another embodiment of the GLP-1 analogue of formula II, Xaa at position 9 is Glu.

In another embodiment of the GLP-1 analogue of formula II, Xaa at position 91 is Thr.

In another embodiment of the GLP-1 analogue of formula II, Xaa at position 14 is Ser.

In another embodiment of the GLP-1 analogue of formula II, Xaa at position 16 is Val.

In another embodiment of the GLP-1 analogue of formula II, Xaa at position 17 is Ser.

In another embodiment of the GLP-1 analogue of formula II, Xaa at position 18 is Ser, Lys, Glu, or Asp.

In another embodiment of the GLP-1 analogue of formula II, Xaa at position 19 is Tyr, Lys, Glu, or Asp.

In another embodiment of the GLP-1 analogue of formula II, Xaa at position 20 is Leu, Lys, Glu, or Asp.

In another embodiment of the GLP-1 analogue of formula II, Xaa at position 21 is Glu, Lys, or Asp.

In another embodiment of the GLP-1 analogue of formula II, Xaa at position 22 is Gly, Glu, Asp, or Lys.

In another embodiment of the GLP-1 analogue of formula II, Xaa at position 23 is Gln, Glu, Asp, or Lys.

In another embodiment of the GLP-1 analogue of formula II, Xaa at position 24 is Ala, Glu, Asp, or Lys.

In another embodiment of the GLP-1 analogue of formula II, Xaa at position 25 is Ala, Glu, Asp, or Lys.

In another embodiment of the GLP-1 analogue of formula II, Xaa at position 26 is Lys, Glu, Asp, or Arg.

In another embodiment of the GLP-1 analogue of formula II, Xaa at position 27 is Glu, Asp, or Lys.

In another embodiment of the GLP-1 analogue of formula II, Xaa at position 30 is Ala, Glu, Asp, or Lys.

In another embodiment of the GLP-1 analogue of formula II, Xaa at position 31 is Trp, Glu, Asp, or Lys.

In another embodiment of the GLP-1 analogue of formula II, Xaa at position 32 is Leu, Glu, Asp, or Lys.

In another embodiment of the GLP-1 analogue of formula II, Xaa at position 33 is Val, Glu, Asp, or Lys.

In another embodiment of the GLP-1 analogue of formula II, Xaa at position 34 is Lys, Arg, Glu, or Asp.

In another embodiment of the GLP-1 analogue of formula II, Xaa at position 35 is Gly, Glu, Asp, or Lys.

In another embodiment of the GLP-1 analogue of formula II, Xaa at position 36 is Arg, Lys, Glu, or Asp.

In another embodiment of the GLP-1 analogue of formula II, Xaa at position 37 is Gly, Glu, Asp, or Lys.

In another embodiment of the GLP-1 analogue of formula II, Xaa at position 38 is Arg, or Lys, or is deleted.

In another embodiment of the GLP-1 analogue of formula II, Xaa at position 39 is deleted.

In another embodiment of the GLP-1 analogue of formula II, Xaa at position 40 is deleted.

In another embodiment of the GLP-1 analogue of formula II, Xaa at position 41 is deleted.

In another embodiment of the GLP-1 analogue of formula II, Xaa at position 42 is deleted.

In another embodiment of the GLP-1 analogue of formula II, Xaa at position 43 is deleted.

In another embodiment of the GLP-1 analogue of formula II, Xaa at position 44 is deleted.

In another embodiment of the GLP-1 analogue of formula II, Xaa at position 45 is deleted.

In another embodiment of the GLP-1 analogue of formula II, Xaa at position 26 is Arg, each of Xaa at positions 37-45 is deleted, and each of the other Xaa is the amino acid in native GLP-1(7-36).

In another embodiment of the GLP-1 analogue of formula II, Xaa at position 26 is Arg, each of Xaa at positions 38-45 is deleted, and each of the other Xaa is the amino acid in native GLP-1(7-37).

In another embodiment of the GLP-1 analogue of formula II, Xaa at position 26 is Arg, each of Xaa at positions 39-45 is deleted, and each of the other Xaa is the amino acid in native GLP-1(7-38).

In another embodiment of the GLP-1 analogue of formula II, Xaa at position 34 is Arg, each of Xaa at positions 37-45 is deleted, and each of the other Xaa is the amino acid in native GLP-1(7-36).

In another embodiment of the GLP-1 analogue of formula II, Xaa at position 34 is Arg, each of Xaa at positions 38-45 is deleted, and each of the other Xaa is the amino acid in native GLP-1(7-37).

In another embodiment of the GLP-1 analogue of formula II, Xaa at position 34 is Arg, each of Xaa at positions 39-45 is deleted, and each of the other Xaa is the amino acid in native GLP-1(7-38).

In another embodiment of the GLP-1 analogue of formula II, Xaa at positions 26 and 34 is Arg, Xaa at position 36 is Lys, each of Xaa at positions 37-45 is deleted, and each of the other Xaa is the amino acid in native GLP-1(7-36).

In another embodiment of the GLP-1 analogue of formula II, Xaa at positions 26 and 34 is Arg, Xaa at position 36 is Lys, each of Xaa at positions 38-45 is deleted, and each of the other Xaa is the amino acid in native GLP-1(7-37).

In another embodiment of the GLP-1 analogue of formula II, Xaa at positions 26 and 34 is Arg, Xaa at position 36 is Lys, each of Xaa at positions 39-45 is deleted, and each of the other Xaa is the amino acid in native GLP-1(7-38).

In another embodiment of the GLP-1 analogue of formula II, Xaa at positions 26 and 34 is Arg, Xaa at position 38 is Lys, each of Xaa at positions 39-45 is deleted, and each of the other Xaa is the amino acid in native GLP-1(7-38).

In another embodiment of the GLP-1 analogue of formula II, Xaa at position 8 is Thr, Ser, Gly, or Val, Xaa at position 37 is Glu, Xaa at position 36 is Lys, each of Xaa at positions 38-45 is deleted, and each of the other Xaa is the amino acid in native GLP-1(7-37).

In another embodiment of the GLP-1 analogue of formula II, Xaa at position 8 is Thr, Ser, Gly, or Val, Xaa at position 37 is Glu, Xaa at position 36 is Lys, each of Xaa at positions 39-45 is deleted, and each of the other Xaa is the amino acid in native GLP-1(7-38).

In another embodiment of the GLP-1 analogue of formula II, Xaa at position 8 is Thr, Ser, Gly, or Val, Xaa at position 37 is Glu, Xaa at position 38 is Lys, each of Xaa at positions 39-45 is deleted, and each of the other Xaa is the amino acid in native GLP-1(7-38).

In another embodiment of the GLP-1 analogue of formula II, Xaa at position 18, 23, or 27 is Lys, and Xaa at positions 26 and 34 is Arg, each of Xaa at positions 37-45 is deleted, and each of the other Xaa is the amino acid in native GLP-1(7-36).

In another embodiment of the GLP-1 analogue of formula II, Xaa at position 18, 23, or 27 is Lys, and Xaa at positions 26 and 34 is Arg, each of Xaa at positions 38-45 is deleted, and each of the other Xaa is the amino acid in native GLP-1(7-37).

In another embodiment of the GLP-1 analogue of formula II, Xaa at position 18, 23, or 27 is Lys, and Xaa at positions 26 and 34 is Arg, each of Xaa at positions 39-45 is deleted, and each of the other Xaa is the amino acid in native GLP-1(7-38).

In another embodiment of the GLP-1 analogue of formula II, Xaa at position 8 is Thr, Ser, Gly, or Val, Xaa at position 18, 23, or 27 is Lys, and Xaa at position 26 and 34 is Arg, each of Xaa at positions 37-45 is deleted, and each of the other Xaa is the amino acid in native GLP-1(7-36).

In another embodiment of the GLP-1 analogue of formula II, Xaa at position 8 is Thr, Ser, Gly, or Val, Xaa at position 18, 23, or 27 is Lys, and Xaa at position 26 and 34 is Arg, each of Xaa at positions 38-45 is deleted, and each of the other Xaa is the amino acid in native GLP-1(7-37).

In another embodiment of the GLP-1 analogue of formula II, Xaa at position 8 is Thr, Ser, Gly, or Val, Xaa at position 18, 23, or 27 is Lys, and Xaa at position 26 and 34 is Arg, each of Xaa at positions 39-45 is deleted, and each of the other Xaa is the amino acid in native GLP-1(7-38).

Such GLP-1 analogues of formula 11 includes, but is not limited to, Arg²⁶-GLP-1(7-37); Arg³⁴-GLP-1(7-37); Lys³⁶-GLP-1(7-37); Arg^{26,34}Lys³⁶-GLP-1(7-37); Arg^{26,34}Lys³⁸GLP-1(7-38); Arg^{26,34} Lys³⁹-GLP-1(7-39); Arg^{26,34}Lys⁴⁰-GLP-1(7-40); Arg²⁶Lys³⁶ -GLP-1(7-37); Arg³⁴Lys³⁶ -GLP-1(7-37); Arg²⁶Lys³⁹-GLP-1(7-39); Arg³⁴Lys⁴⁰-GLP-1(7-40); Arg^{26,34}Lys^{36,39}-GLP-1(7-39); Arg^{26,34}Lys^{36,40}-GLP-1(7-40); Gly⁸Arg26-GLP-1(7-37); Gly⁸Arg³⁴-GLP-1(7-37); Val⁸-GLP-1(7-37); Thr⁸-GLP-1(7-37); Gly⁸-GLP-1(7-37); Met⁸-GLP-1(7-37); Gly⁸Lys³⁶-GLP-1(7-37); Gly⁸Arg^{26,34}Lys³⁶-GLP-1(7-37); Gly⁸Arg^{26,34}Lys³⁹-GLP-1(7-39); Gly⁸Arg^{26,34}Lys⁴⁰-GLP-1(7-40); Gly⁸Arg²⁶Lys³⁶-GLP-1(7-37); Gly⁸Arg³⁴Lys³⁶-GLP-1(7-37); Gly⁸Arg²⁶Lys³⁹-GLP-1(7-39); Gly⁸Arg³⁴Lys⁴⁰-GLP-1(7-40); Gly⁸Arg^{26,34}Lys^{36,39}-GLP-1(7-39); Gly⁸Arg^{26,34}Lys^{36,40}-GLP-1(7-40); Arg^{26,34}Lys³⁸GLP-1(7-38); Arg^{26,34}Lys³⁹GLP-1(7-39); Arg^{26,34}Lys⁴⁰GLP- 1(7-40); Arg^{26,34}Lys⁴¹GLP-1(7-41); Arg^{26,34}Lys⁴²GLP-1(7-42); Arg^{26,34}Lys⁴³GLP-1(7-43); Arg^{26,34}Lys⁴⁴GLP-1(7-44); Arg^{26,34}Lys⁴⁵GLP-1(7-45); Arg^{26,34}Lys³⁸GLP-1(1-38); Arg^{26,34}Lys³⁹GLP-1(1-39); Arg^{26,34}Lys⁴⁰GLP-1(1-40); Arg^{26,34}Lys⁴¹GLP-1(2-41); Arg^{26,34}Lys⁴²GLP-1(1-42); Arg^{26,34}Lys⁴³GLP-1(1-43); Arg^{26,34}Lys⁴⁴GLP-1(1-44); Arg^{26,34}Lys⁴⁵GLP-1(1-45); Arg^{26,34}Lys³⁸GLP-1(2-38); Arg^{26,34}Lys³⁹GLP-1(2-39); Arg^{26,34}Lys⁴⁰GLP-1(2-40); Arg^{26,34}Lys⁴¹GLP-1(2-41); Arg^{26,34}Lys⁴²GLP-1(2-42); Arg^{26,34}Lys⁴³GLP-1(2-43); Arg^{26,34}Lys⁴⁴GLP-1(2-44); Arg^{26,34}Lys⁴⁵GLP-1(2-45); Arg^{26,34}Lys³⁸GLP-1(3-38); Arg^{26,34}Lys³⁹GLP-1(3-39); Arg^{26,34}Lys⁴⁰GLP-1(3-40); Arg^{26,34}Lys⁴¹GLP-1(3-41); Arg^{26,34}Lys⁴²GLP-1(3-42); Arg^{26,34}Lys⁴³GLP-1(3-43); Arg^{26,34}Lys⁴⁴GLP-1(3-44); Arg^{26,34}Lys⁴⁵GLP-1(3-45); Arg^{26,34}Lys³⁸GLP-1(4-38); Arg^{26,34}Lys³⁹GLP-1(4-39); Arg^{26,34}Lys⁴⁰GLP-1(4-40); Arg^{26,34}Lys⁴¹GLP-1(4-41); Arg^{26,34}Lys⁴²GLP-1(4-42); Arg^{26,34}Lys⁴³GLP-1(4-43); Arg^{26,34}Lys⁴⁴GLP-1(4-44); Arg^{26,34}Lys⁴⁵GLP-1(4-45); Arg^{26,34}Lys³⁸GLP-1(5-38); Arg^{26,34}Lys³⁹GLP-1(5-39); Arg^{26,34}Lys⁴⁰GLP-1(5-40); Arg^{26,34}Lys⁴¹GLP-1(5-41); Arg^{26,34}Lys⁴²GLP-1(5-42); Arg^{26,34}Lys⁴³GLP-1(5-43); Arg^{26,34}Lys⁴⁴GLP 1(5-44); Arg^{26,34}Lys⁴⁵GLP-1(5-45); Arg^{26,34}Lys⁴²GLP-1(6-38); Arg^{26,34}Lys³⁹GLP-1(6-39); Arg^{26,34}Lys⁴⁰GLP-1(6-40); Arg^{26,34}Lys⁴¹GLP-1(6-41); Arg^{26,34}Lys⁴²GLP-1(6-42); Arg^{26,34}Lys⁴³GLP-1(6-43); Arg^{26,34}Lys⁴⁴GLP-1(6-44); Arg^{26,34}Lys⁴⁵GLP-1(6-45); Arg²⁶Lys³⁸GLP-1(1-38); Arg³⁴Lys³⁸GLP-1(1-38); Arg^{26,34}Lys^{36,38}GLP-1(1-38); Arg²⁶Lys³⁸GLP-1(7-38); Arg³⁴Lys³⁸GLP-1(7-38); Arg^{26,34}Lys^{36,38}GLP 1(7-38); Arg^{26,34}Lys³⁸GLP-1(7-38); Arg²⁶Lys³⁹GLP-1(1-39); Arg³⁴Lys³⁹GLP-1(1-39); Arg^{26,34}Lys^{36,39}GLP-1(1-39); Arg²⁶Lys³⁹GLP-1(7-39); Arg³⁴Lys³⁹GLP-1(7-39) and Arg^{26,34}Lys^{36,39}GLP-1(7-39). Each one of these specific GLP-1 analogues constitutes an alternative embodiment of the invention.

In a further embodiment of the invention the GLP-1 analogue has the formula III

A—HN—GLP-1(8-B)—X (III)

wherein

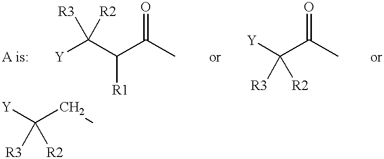

wherein

R¹, R² and R³ are independently H, lower alkyl having 1 to 6 carbon atoms, optionally substituted phenyl, NH₂, NH—CO-(lower alkyl), —OH, lower alkoxy having 1 to 6 carbon atoms, halogen, SO₂-(lower alkyl) or CF₃, said phenyl is optionally substituted with at least one group selected from NH₂, —OH, lower alkyl or lower alkoxy having 1-6 carbon atoms, halogen, SO₂-(lower alkyl), NH—CO-(lower alkyl) or CF₃, or R¹ and R² may together form a bond; Y is a five or six membered ring system selected from the group consisting of:

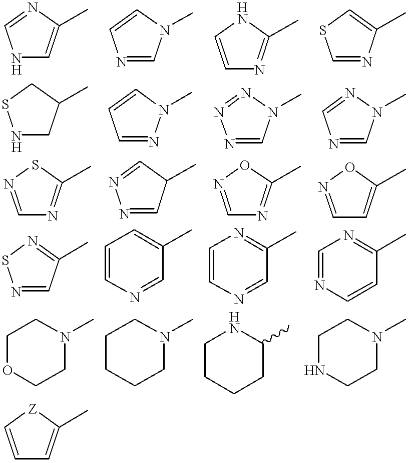

wherein

Z is N, O or S, said ring system is optionally substituted with one or more functional groups selected from the group consisting of NH₂, NO₂, OH, C₁₋₆ alkyl, C₁₋₆ alkoxy, halogen (Cl, Br, F, I), CF,₃ and aryl;

B is an integer in the range of 35-45; and

X is —OH, —NH₂, or a C₁₋₆ alkyl amide or C₁₋₆ dialkyl amide group;

or an analogue thereof.

Such GLP-1 analogues of formula III includes, but is not limited to

Arg²⁶-GLP-1(7-37); Arg³⁴-GLP-1(7-37); Lys³⁶ -GLP-1(7-37);

Arg^{26,34}Lys³⁶-GLP-1(7-37); Arg^{26,34}Lys³⁸GLP-1(7-38);

Arg^{26,34}Lys³⁹-GLP-1(7-39); Arg^{26,34}Lys⁴⁰-GLP-1(7-40);

Arg²⁶Lys³⁶-GLP-1(7-37); Arg³⁴Lys³⁶-GLP-1(7-37);

Arg²⁶Lys³⁹-GLP-1(7-39); Arg³⁴Lys⁴⁰-GLP-1(7-40);

Arg^{26,34}Lys^{36,39}-GLP-1(7-39); Arg^{26,34}Lys^{36,40}-GLP-1(7-40);

Gly⁸Arg²⁶-GLP-1(7-37); Gly⁸Arg³⁴-GLP-1(7-37);

Gly⁸Lys³⁶-GLP-1(7-37); Gly⁸Arg^{26,34}Lys³⁶-GLP-1(7-37);

Gly⁸Arg^{26,34}Lys³⁹-GLP-1(7-39); Gly⁸Arg^{26,34}Lys⁴⁰-GLP-1(7-40);

Gly⁸Arg²⁶Lys³⁶-GLP-1(7-37); Gly⁸Arg³⁴Lys³⁶-GLP-1(7-37);

Gly⁸Arg²⁶Lys³⁹-GLP-1(7-39); Gly⁸Arg³⁴Lys⁴⁰-GLP-1(7-40);

Gly⁸Arg^{26,34}Lys^{36,39}-GLP-1(7-39); or Gly⁸Arg^{26,34}Lys^{36,40}-GLP-1(7-40). Each one of these specific GLP-1 analogues constitutes an alternative embodiment of the invention.

In a further embodiment of the invention the GLP-1 analogue has the formula IV

A—GLP-1(19-B)—X (IV)

wherein

A is a peptide comprising the amino acid residues of GLP-1(8-18) or a fragment thereof;

B is an integer in the range of 35-45; and

X is —OH, —NH₂, or a C₁₋₆ alkyl amide or C₁₋₆ dialkyl amide group; or an analogue thereof.

In an embodiment of the GLP-1 analogue of formula IV, A is a peptide selected from the group consisting of GLP-1(8-18), GLP-1(9-18), GLP-1(10-18), GLP-1(11-18), GLP-1(12-18), GLP-1(13-18), GLP-1(14-18), GLP-1(15-18), GLP-1(16-18), GLP-1(17-18) and GLP-1(18). Preferably, A is GLP-1(8-18), GLP-1(9-18), GLP-1(10-18), GLP-1(11-18) or GLP-1(12-18), and B is 36, 37 or 38. Most preferably, A is GLP-1(8-18).

In a further embodiment of the GLP-1 analogue of formula IV, B is 35, 36, 37, 38, 39, 40, 41, 42, 43 or 44. In a more preferred embodiment, B is 36. In another more preferred embodiment. B is 37. In another more preferred embodiment, B is 38.

Such GLP-1 analogues of formula IV includes, but is not limited to

Arg²⁶-GLP-1(8-37); Arg³⁴-GLP-1(8-37); Lys³⁶-GLP-1(8-37);

Arg^{26,34}Lys³⁶-GLP-1(8-37); Arg^{26,34}Lys³⁸GLP-1(8-38);

Arg^{26,34}Lys³⁹-GLP-1(8-39); Arg^{26,34}Lys⁴⁰-GLP-1(8-40);

Arg²⁶Lys³⁶-GLP-1(8-37); Arg³⁴Lys³⁶-GLP-1(8-37);

Arg²⁶Lys³⁹-GLP-1(8-39); Arg³⁴Lys⁴⁰-GLP-1(8-40);

Arg^{26,34}Lys^{36,39}-GLP-1(8-39); Arg^{26,34}Lys^{36,40}-GLP-1-(8-40);

Gly⁸Arg²⁶-GLP-1(8-37); Gly⁸Arg³⁴-GLP-1(8-37);

Gly⁸Lys³⁶-GLP-1(8-37); Gly⁸Arg^{26,34}Lys³⁶-GLP-1(8-37);

Gly⁸Arg^{26,34}Lys³⁹-GLP-1(8-39); Gly⁸Arg^{26,34}Lys⁴⁰-GLP-1(8-40);

Gly⁸Arg²⁶Lys³⁶-GLP-1(8-37); Gly⁸Arg³⁴Lys³⁶-GLP-1(8-37);

Gly⁸Arg²⁶Lys³⁹-GLP-1(8-39); Gly⁸Arg³⁴Lys⁴⁰-GLP-1(8-40);

Gly⁸Arg^{26,34}Lys^{36,39}-GLP-1(8-39); or

Gly⁸Arg^{26,34}Lys^{36,40}-GLP-1(8-40). Each one of these specific GLP-1 analogues constitutes an alternative embodiment of the invention.

In one embodiment of the present invention the lipophilic substituent comprises 4-40 carbon atoms. In a further embodiment of the present invention the lipophilic substituent comprises 8-30 carbon atoms. In a further embodiment of the present invention the lipophilic substituent comprises 8-25 carbon atoms. In a further embodiment of the present invention the lipophilic substituent comprises 12-25 carbon atoms. In a further embodiment of the present invention the lipophilic substituent comprises 14-18 carbon atoms.

The lipophilic substituent(s) contain a functional group which can be attached to one of the following functional groups of an amino acid of the parent GLP-1 compound:

(a) the amino group attached to the alpha-carbon of the N-terminal amino acid,

(b) the carboxy group attached to the alpha-carbon of the C-terminal amino acid,

(c) the epsilon-amino group of any Lys residue,

(d) the carboxy group of the R group of any Asp and Glu residue,

(e) the hydroxy group of the R group of any Tyr, Ser and Thr residue,

(f) the amino group of the R group of any Trp, Asn, Gln, Arg, and His residue, or

(g) the thiol group of the R group of any Cys residue.

In an embodiment, a lipophilic substituent is attached to the carboxy group of the R group of any Asp and Glu residue.

In another embodiment, a lipophilic substituent is attached to the carboxy group attached to the alpha-carbon of the C-terminal amino acid.

In a most preferred embodiment, a lipophilic substituent is attached to the epsilon-amino group of any Lys residue.

Each lipophilic substituent contains a functional group which may be attached to a functional group of an amino acid of the parent GLP-1 compound. For example, a lipophilic substituent may contain a carboxyl group which can be attached to an amino group of the parent GLP-1 peptide by means of an amide bond.

In a further embodiment, the lipophilic substituent comprises a partially or completely hydrogenated cyclopentanophenathrene skeleton.

In another embodiment, the lipophilic substituent is a straight-chain or branched alkyl group.

In another embodiment, the lipophilic substituent is an acyl group of a straight-chain or branched fatty acid.

In a further embodiment the lipophilic substituent is an acyl group having the formula CH₃(CH₂)ₙCO—, wherein n is an integer from 4 to 38. In a further embodiment n is an integer from 12 to 38. In further embodiments the lipophilic substituent is selected from the following individual embodiments CH₃(CH₂)₁₂CO—, CH₃(CH₂)₁₄CO—, CH₃(CH₂)₁₆CO—, CH₃(CH₂)₁₈CO—, CH₃(CH₂)₂₀CO—, and CH₃(CH₂)₂₂CO—. In a specific embodiment, the lipophilic substituent is tetradecanoyl. In another specific embodiment, the lipophilic substituent is hexadecanoyl.

In another embodiment of the present invention, the lipophilic substituent has a group which is negatively charged such as a carboxylic acid group. For example, the lipophilic substituent may be an acyl group of a straight-chain or branched alkane α,ω-dicarboxylic acid of the formula HOOC(CH₂)ₘCO—, wherein m is an integer from 4 to 38, preferably an integer from 12 to 38, and most preferably is HOOC(CH₂)₁₄CO—, HOOC(CH₂)₁₆CO—, HOOC(CH₂)₁₈CO—, HOOC(CH₂)₂₀CO—, or HOOC(CH₂)₂₂CO—.

In a further embodiment of the invention, the lipophilic substituent is attached to the parent GLP-1 compound by means of a spacer. A spacer must contain at least two functional groups, one to attach to a functional group of the lipophilic substituent and the other to a functional group of the parent GLP-1 compound.

In an embodiment, the spacer is an amino acid residue except Cys or Met. In another embodiment, the spacer is a dipeptide such as Gly-Lys.

In a further embodiment the spacer is selected from lysyl, glutamyl, asparagyl, glycyl, beta-alanyl and gamma-aminobutanoyl, each of which constitutes an individual embodiment. Most preferred spacers are glutamyl and beta-alanyl.

In another embodiment, the spacer is an unbranched alkane α,ω-dicarboxylic acid group having from 1 to 7 methylene groups, which spacer forms a bridge between an amino group of the parent peptide and an amino group of the lipophilic substituent. Preferably, the spacer is succinic acid.

In a further embodiment, the lipophilic substituent with the attached spacer is a group of the formula CH₃(CH₂)ₚNH—CO(CH₂)_{q}CO—, wherein p is an integer from 8 to 33, such as from 12 to 28 and q is an integer from 1 to 6, such as 2.

In a further embodiment, the lipophilic substituent with the attached spacer is a group of the formula CH₃(CH₂)ᵣCO—NHCH(COOH)(CH₂)₂CO—, wherein r is an integer from 4 to 24, such as from 10 to 24.

In a further embodiment, the lipophilic substituent with the attached spacer is a group of the formula CH₃(CH₂)ₛCO—NHCH((CH₂)₂COOH)CO—, wherein s is an integer from 4 to 24, preferably from 10 to 24.

In a further embodiment, the lipophilic substituent is a group of the formula COOH(CH₂)ₜCO— wherein t is an integer from 6 to 24.

In a further embodiment, the lipophilic substituent with the attached spacer is a group of the formula —NHCH(COOH)(CH₂)₄NH—CO(CH₂)ᵤCH₃, wherein u is an integer from 8 to 18.

In a further embodiment, the lipophilic substituent with the attached spacer is a group of the formula CH₃(CH₂)ᵥCO—NH—(CH₂)_{z}—CO, wherein v is an integer from 4 to 24 and z is an integer from 1 to 6.

In a further embodiment, the lipophilic substituent with the attached spacer is a group of the formula —NHCH(COOH)(CH₂)₄NH—COCH((CH₂)₂COOH)NH—CO(CH₂)_{w}CH₃, wherein w is an integer from 10 to 16.

In a further embodiment, the lipophilic substituent with the attached spacer is a group of the formula —NHCH(COOH)(CH₂)₄NH—CO(CH₂)₂CH(COOH)NHCO(CH₂)ₓCH₃, wherein x is zero or an integer from 1 to 22, such as from 10 to 16.

The term “GLP-1” means GLP-1(7-37) or GLP-1(7-36) amide. The amino acid sequence of GLP-1 is given i.a. by Schmidt et al. (Diabetologia 28 704-707(1985).

The term “treatment” is defined as the management and care of a patient, e.g. a mammal, in particular a human, for the purpose of combating the disease, condition, or disorder and includes the administration of a GLP-1 compound to prevent the onset of the symptoms or complications, or alleviating the symptoms or complications, or eliminating the disease, condition, or disorder.

In the present context “a GLP-1 compound” is intended to indicate GLP-1 or an analogue or a derivative thereof, or exendin or an analogue or a derivative thereof, which binds to a GLP-1 receptor, preferably with an affinity constant, K_{D}, below 1 μM, e.g. below 100 nM. Methods for identifying GLP-1 compounds are described in WO 93/19175 (Novo Nordisk A/S). Suitable GLP-1 compounds have been disclosed in e.g. WO 87/06941, WO 90/11296, WO 93/25579, WO 91/11457, EP 0699686, WO 98/43658, EP 0619322, which are incorporated herein by reference.

In the present context “a GLP-1 compound” is also intended to comprise active metabolites and prodrugs thereof, such as active metabolites and prodrugs of GLP-1 or an analogue or a derivative thereof, or exendin or an analogue or a derivative thereof. A “metabolite” is an active derivative of a GLP-1 compound produced when the GLP-1 compound is metabolized. A “prodrug” is a compound which is either metabolized to a GLP-1 compound or is metabolized to the same metabolite(s) as a GLP-1 compound.

In the present text, the designation “an analogue” is used to designate a peptide wherein one or more amino acid residues of the parent peptide have been substituted by another amino acid residue and/or wherein one or more amino acid residues of the parent peptide have been deleted and/or wherein one or more amino acid residues have been added to the parent peptide. Such addition can take place either in the peptide, at the N-terminal end or at the C-terminal end of the parent peptide, or any combination thereof.

The term “derivative” is used in the present text to designate a peptide in which one or more of the amino acid residues of the parent peptide have been chemically modified, e.g. by alkylation, acylation, ester formation or amide formation.

The term “lipophilic substituent” is characterised by comprising 4-40 carbon atoms and having a solubility in water at 20° C. in the range from about 0.1 mg/100 ml water to about 250 mg/100 ml water, preferable in the range from about 0.3 mg/100 ml water to about 75 mg/100 ml water. For instance, octanoic acid (C8) has a solubility in water at 20° C. of 68 mg/100 ml, decanoic acid (C10) has a solubility in water at 20° C. of 15 mg/100 ml, and octa-decanoic acid (C18) has a solubility in water at 20° C. of 0.3 mg/100 ml.

The term “spacer” is used in the present text to designate a bivalent moiety which contain at least two functional groups, one to attach to a functional group of the lipophilic substituent and the other to a functional group of the GLP-1 compound. Examples of suitable spacers are succinic acid, lysyl, glutamyl, asparagyl, glycyl, beta-alanyl and gamma-aminobutanoyl, or a dipeptide such as Gly-Lys.

The term “an effective amount” is the effective dose to be determined by a qualified practitioner, who may titrate dosages to achieve the desired response. Factors for consideration of dose will include potency, bioavailability, desired pharmacokinetic/pharmacodynamic profiles, condition of treatment (e.g. diabetes, obesity, gastric ulcers), patient-related factors (e.g. weight, health, age, etc.), presence of co-administered medications (e.g. insulin), time of administration, or other factors known to a medical practitioner.

In one embodiment, an effective amount of a GLP-1 compound whereto is attached a lipophilic substituent optionally via a spacer will range from 0.01-1000.0 μg/kg, more preferably from 0.05-500 μg/kg, even more preferably from 0.1-100 μg/kg, such as from 0.5-50 μg/kg .

Ranges of required daily doses would typically include the equivalent of about 0.1-4.0 inhalations/day. In a preferred embodiment, the inhaled GLP-1 compound whereto is attached a lipophilic substituent optionally via a spacer is administered once daily and demonstrate bioequivalence to a sub-cutaneous dosage form.

Any possible combination of two or more of the embodiments described herein, is comprised within the scope of the present invention.

In an embodiment the pulmonary formulation is a liquid formulation, such as a solution or a suspension.

In another embodiment the pulmonary formulation is a dry formulation.

In an embodiment the pulmonary delivery device is selected from nebulizers, such as jet or ultrasonic nebulizers, metered-dose inhalers, or dry powder inhalers. Each of said devices is considered an individual embodiment.

Ideally, aerosol formulations for pulmonary delivery of a GLP-1 compound whereto is attached a lipophilic substituent optionally via a spacer could be designed which minimise the excipient requirements and maximise bioactive delivery of a GLP-1 compound whereto is attached a lipophilic substituent optionally via a spacer to the deep lung (i.e. alveolar tissue), where compounds are known to be predominantly absorbed (cf. Yu J, Chien Y W. Pulmonary drug delivery: Physiologic and mechanistic aspects. Crit Rev Ther Drug Carr Sys 14(4) (1997) 395-453). Aside from the basic demands of safety and efficacy, said formulations should also be designed in accordance with common pharmaceutical development goals and have features such as, long-term stability and preservation from bacterial or fungal contamination during defined shelf-lives. Additionally, formulations should allow for controlled delivery profiles in order to optimise the pharmacokinetic/pharmacodynamic profile following in vivo pulmonary delivery of a GLP-1 compound whereto is attached a lipophilic substituent optionally via a spacer.

The GLP-1 compound whereto is attached a lipophilic substituent optionally via a spacer may be produced by any recognised peptide/protein synthetic, semi-synthetic and/or recombinant DNA techniques.

The GLP-1 compound whereto is attached a lipophilic substituent optionally via a spacer can be delivered in a vehicle, as a solution, suspension, or dry powder and can be administered by any of the known devices suitable for pulmonary drug delivery known in the art. Ideally, a GLP-1 compound whereto is attached a lipophilic substituent optionally via a spacer can be administered by any of three general types of aerosol-generating systems for pulmonary drug delivery, and include jet or ultrasonic nebulizers, metered-dose inhalers, or dry powder inhalers (Cf. Yu J, Chien Y W. Pulmonary drug delivery: Physiologic and mechanistic aspects. Crit Rev Ther Drug Carr Sys 14(4) (1997) 395-453).

The terms “MMAD” and “MMEAD” are well-described and known to the art (cf. Edwards D A, Ben-Jebria A, Langer R. Recent advances in pulmonary drug delivery using large, porous inhaled particles. J Appl Physiol 84(2) (1998) 379-385). Mass median aerodynamic diameter (MMAD) and mass median effective aerodynamic diameter (MMEAD) are used inter-changeably, are statistical parameters, and empirically describe the size of aerosol particles in relation to their potential to deposit in the lungs, independent of actual shape, size, or density (cf. Edwards D A, Ben-Jebria A, Langer R. Recent advances in pulmonary drug delivery using large, porous inhaled particles. J Appl Physiol 84(2) (1998) 379-385).

Based on standardised testing methodology, the aerodynamic diameter (dₐ) of a particle is defined as the geometric equivalent diameter of a reference standard spherical particle of unit density (1 g/cm³). In the simplest case, for spherical particles, dₐ is related to a reference diameter (d) as a function of the square root of the density ratio as described by:

$${d}_{a} = \sqrt{\frac{ρ}{{ρ}_{a}}} d$$

Modifications to this relationship occur for non-spherical particles (cf. Edwards D A, Ben-Jebria A, Langer R. Recent advances in pulmonary drug delivery using large, porous inhaled particles. J Appl Physiol 84(2) (1998) 379-385). MMAD is normally measured by cascade impactors, which estimate the particle size based on the particle behavior in a high velocity airstream. It is commonly accepted that a MMAD window between 1-3 μm is optimal for deposition of particles in the deep lung.

In a further embodiment stable liquid formulations of GLP-1 compounds whereto is attached a lipophilic substituent optionally via a spacer can be designed for nebulisation. Such liquid formulations may contain preservative agents, isotonicty agents, buffering agents, antioxidants, flavorants, or delivery modifying agents so as to improve the shelf-life and performance of formulated products.

Preservatives may be required to develop a commercial product for multiple-use. Preservatives may include, but are not limited to, phenolics, such as phenol or m-cresol, benzyl alcohol, chlorobutanol, parabens, quaternary ammonium compounds, thirmerosal, or phenylmercuric salts or combinations thereof. Phenol or m-cresol at concentrations between 2-5 mg/mL is preferred as a preservative agent.

Pharmaceutically acceptable isotonicity agents may include NaCl, dextrose, mannitol, lactose, or glycerin.

Pharmaceutically acceptable buffering agents for controlling formulation pH may include, but are not limited to, phosphates, citrates, acetates, TRIS, amino acids, or amino acid based salts (e.g. glycylglycine).

Pharmaceutically acceptable antioxidants may be included to improve the chemical stability profile of the GLP-1 compound whereto is attached a lipophilic substituent optionally via a spacer. Suitable antioxidants may include, but are not limited to, phenolic compounds (e.g.BHT, BHA, popyl gallate, α-tocopherol), reducing agents (e.g. methionine, ascorbic acid, sodium sulfite, thioglycerol, thioglycolic acid), or chelating agents (e.g. EDTA, citric acid, or thioglycolic acid).

In a further embodiment delivery modifying agents, in this context, can include substances which can be added to the formulation in order to improve delivery efficiency of the GLP-1 compound whereto is attached a lipophilic substituent optionally via a spacer to the lower lung, or modify the permeation of the GLP-1 compound whereto is attached a lipophilic substituent optionally via a spacer across the pulmonary epithelium. In this context, the added ingredient may: 1) on exposure to an aerosol-generating device, facilitate nebulisation of liquids to achieve particle sizes within the optimum window of 1-3 μm MMAD, which has been defined as being optimal for deep lung deposition (cf. Edwards D A, Ben-Jebria A, Langer R. Recent advances in pulmonary drug delivery using large, porous inhaled particles. J Appl Physiol 84(2) (1998) 379-385), 2) maximise aerosolisation and delivery of protein out of the aerosol-generating device, by, for example, preventing losses associated with protein adsorption to device surfaces, 3) modify the aggregation state of the solution stable peptide which can modulate the permeability characteristics of the peptide across pulmonary epithelia. Examples of delivery modifying agents include, but are not limited to, complexing agents (e.g. divalent metals, cyclodextrins, proteins (e.g. albumin, protamine)), phospholipids, glycolipids, glycerides, carbohydrates, surfactants, viscosity modifying agents (e.g. glycerol, glycols, hydrophilic biocompatible polymers (e.g. polyethylene glycols, pluronics, methylcellulose derivatives, carbopols, chitosans, etc.)), semi-polar co-solvents (e.g. ethanol), salts, or, alternatively, small organic molecules as described by Emisphere Technologies (cf. WO 98/25589 Active agent transport systems). Choice and amount of excipients in the formulation would depend primarily on the excipients safety record (i.e. toxicological profile). This safety record would include both systemic and local pulmonary toxicity determinations. Advantageously, no added excipient will adversely affect the airways of the patient.

Typical formulations for nebulisation would normally include between 0.1-100 mg of the GLP-1 compound whereto is attached a lipophilic substituent optionally via a spacer per mL solution. More preferably between 1-50 mg protein per mL solution.

One liquid formulation for nebulization could include a GLP-1 compound whereto is attached a lipophilic substituent optionally via a spacer at 5 mg/mL, phenol at 5.0 mg/mL, mannitol at 38 mg/mL, and phosphate buffer adjusted to about pH 7.4 in bacteriostatic water for injection.

In a further embodiment, the liquid formulation could be nebulised by any known nebulisation technology, such as jet or ultrasonic nebulisation, to achieve a MMAD of aerosol particles less than 10 μm, more preferably between 1-5 μm, and most preferably between 1-3 μm. An example of a clinically useful nebuliser could be the Maxin® nebuliser developed by Clinova Medical AB (Malmo, Swe). Ideally, the particle distribution is substantially narrow so as to provide an optimal, reproducible delivery of the GLP-1 compound whereto is attached a lipophilic substituent optionally via a spacer to the lung. The preferred particle size range is based on the most effective size range for delivery of drug to the deep lung, where protein is optimally absorbed. In one embodiment, advanced nebulisation techniques such as those provided for by Aradigm Corp. (AERx® system) could be utilised (cf. U.S. Pat. No. 5,934,272; U.S. Pat. No. 5,855,564).

In an alternative embodiment, the GLP-1 compound whereto is attached a lipophilic substituent optionally via a spacer could be formulated as a dry powder for inhalation. Dry powders have the advantages of room-temperature stability, and high drug payload (e.g. dry powder aerosols contain between 50-95% pure drug) when compared to aqueous formulations for nebulisation, or metered dose inhalers (MDIs), an additional advantage is that DPIs (dry powder inhalers) do not require the co-ordination necessary to operate traditional MDIs (metered dose inhalers) since most are breath activated and are optimally designed to deliver consistent doses independent of inspiratory flow rates over a wide range. This translates to the ability to delivery a much larger amount of drug per patient breath (5-100 fold) when compared MDIs and nebulised solutions. As well, the risk of microbial or fungal contamination is reduced due to formulations being in the dry state. Dry powder formulations can be designed to be highly soluble in pulmonary fluid. Alternatively, controlled pulmonary delivery may be achieved by modifying the solubility of the dry powder formulation, modifying the aggregation state of the solubilized GLP-1 compound whereto is attached a lipophilic substituent optionally via a spacer, or the dry powder particle size. Additives may be included to facilitate controlled pulmonary delivery, processing and filling of powders, aersolisation efficiency of the powder, chemical stabilization, or to provide cosmetic appeal (e.g. flavorants).

Examples of processing, filling, and metering methods for developing dry powders for inhalation are provided for in e.g. U.S. Pat. No. 5,874,064, U.S. Pat. No. 5,855,913, WO9829096, WO9829098, WO9829140, WO9829141, WO9816205, WO9741833, WO97/41833, U.S. Pat. No. 5,780,014, WO 99/16419, U.S. Pat. No. 5,699,649, U.S. Pat. No. 5,654,007, WO 97/47286, WO 98/13031, U.S. Pat. No. 5725841, WO 98/34596, WO 99/36334, WO 98/35888, WO 98/30262.

Examples of excipients can include, but are not limited to, substances which can be added to the formulation in order to improve dry powder processing, metering, and filling, delivery efficiency of the GLP-1 compound whereto is attached a lipophilic substituent optionally via a spacer to the lower lung, or modify the permeation of the GLP-1 compound whereto is attached a lipophilic substituent optionally via a spacer across the pulmonary epithelium. In this context, the added ingredient may: 1) facilitate processing of dry powders to achieve particle sizes within the optimum window of 1-3 μm MMAD, which has been defined as being optimal for deep lung deposition (cf. Edwards D A, Ben-Jebria A, Langer R. Recent advances in pulmonary drug delivery using large, porous inhaled particles. J Appl Physiol 84(2) (1998) 379-385), 2) on exposure to an aerosol-generating device, facilitate the formation of substantially dispersed aerosol particles from a powder cake within the optimum window of 1-3 μm MMAD, 3) maximise aerosolisation and delivery of protein out of the aerosol-generating device, by, for example, preventing losses associated with powder aggregation, 4) improve powder flow characteristics to optimise filling procedures (e.g. bulking agents), 5) modify the aggregation state of the solution stable peptide which can modulate the permeability characteristics of the peptide across pulmonary epithelia, 6) control the release of the GLP-1 compound whereto is attached a lipophilic substituent. Examples of such excipients include, but are not limited to, complexing agents (e.g. divalent metals, cyclodextrins, proteins/polypeptides (e.g. albumin, protamine)), phospholipids, glycolipids, glycerides, carbohydrates, surfactants, biocompatible polymers (polyethylene glycols, PLGA derivatives, pluronics, methylcellulose derivatives, etc.), salts, amino acids, or, alternatively, small organic molecules as described by Emisphere Technologies (cf. WO 98/25589). Choice and amount of excipients in the formulation would depend primarily on the excipients safety record (i.e. toxicological profile). This safety record would be based on relative systemic and local pulmonary toxicity determinations. Advantageously, no added excipient will adversely affect the airways of a patient.

Additionally, antioxidants may be added to prevent chemical degradation of the GLP-1 compound whereto is attached a lipophilic substituent optionally via a spacer. Suitable antioxidants may include, but are not limited to, phenolic compounds (e.g.BHT, BHA, popyl gallate, α-tocopherol), reducing agents (e.g. methionine, ascorbic acid, sodium sulfite, thioglycerol, thioglycolic acid), or chelating agents (e.g. EDTA, citric acid, or thioglycolic acid).

Ideally, dry powders for inhalation would contain between 50-100 %, more preferably between 75-100%, and most preferably between 90-100% GLP-1 compound whereto is attached a lipophilic substituent optionally via a spacer on a w/w basis. Furthermore, the dry powder formulation should be designed to contain a MMAD of aerosol particles less than 10 μm, more preferably between 1-5 μm, and most preferably between 1-3 μm. Ideally, the particle distribution is substantially narrow so as to provide an optimal, reproducible delivery of GLP-1 compound whereto is attached a lipophilic substituent optionally via a spacer to the lung. The preferred particle size range is based on the most effective size range for delivery of drug to the deep lung, where protein is optimally absorbed. The defined optimal particle size range of the protein powders may be obtained by any conventional method know to those skilled in the art, such as spray-drying, spray-coating, jet-milling, extrusion, micronization, lyophilisation, solution condensation, or the like.

The above particles may be supplied to the aerosol-generating device as redispersable aggregates or agglomerates in order to improve the powder handling characteristics, for example during filling of unit dose blister packs. Aggregates, agglomerates, or granules may be formed by techniques known in the art, for example formation of a wetted particle mass with a binding solvent, extrusion of wetted mass through fine mesh screens (ca. 40-650 μm), and subsequent drying, sieving, and optional spheronization steps. Examples of such processes used in protein formulations are provided for in e.g. WO 99/48476, U.S. Pat. No. 5,780,014 and U.S. Pat. No. 5,654,007, and are recognised in the art. Formation of aggregates, agglomerates, granules or the like may include the use of non aqueous solvents such as, a flurocarbon (e.g. perfluorodecalin, perfluorooctylbromide), toluene, xylene, benzene, acetone, hexane, octane, chloroform and methylene chloride.

Packaging of drug product is typically done in unit dose blisters or cartridges, and is completed by techniques know in the art.

Embodiments of devices suitable for dry powder pulmonary delivery of a GLP-1 compound whereto is attached a lipophilic substituent optionally via a spacer include, but are not limited to, devices provided for by 3M, Inhale Therapeutic Systems, Advanced Inhalation Technology Corp., Dura Pharmaceuticals (e.g. Spiros® device), Astra Pharmaceuticals (e.g Turbuhaler® device), Glaxo (e.g. rotahaler® or diskhaler® device), Fisons (e.g. spinhaler® device) or MicroDose Technologies, of which some examples are provided for in e.g. WO 96/32149, U.S. Pat. No. 5,655,523, U.S. Pat. No. 5,645,051, U.S. Pat. No. 5,622,166, U.S. Pat. No. 5,577,497, U.S. Pat. No. 5,492,112, U.S. Pat. No. 5,327,883, U.S. Pat. No. 5,277,195 and U.S. Pat. No. 5,694,920.

In an alternative embodiment, the GLP-1 compound whereto is attached a lipophilic substituent optionally via a spacer may be formulated for use with conventional metered dose inhalers (MDIs). MDIs can usually deliver higher concentrations of active over shorter periods of time when compared to nebulised solutions.

Formulations prepared for MDIs are typically finely dispersed powders, which are suspended in non-aqueous propellant solutions. Alternatively, a solution aerosol can be made by including organic co-solvents, such as ethanol. Propellents used can be chosen from common materials such as, chlorofluorcarbons, hydrochlorofluorocarbons, hydroflurocarbons, or hydrocarbons. Preferably the propellant is chosen to be more environmentally friendly, such as the hydrofluorocarbons. The use of additional excipients may be necessary to stabilise the dispersed powder suspension, to prevent chemical degradation, or to optimise the delivery of the GLP-1 compound whereto is attached a lipophilic substituent optionally via a spacer in a finely dispersed form. The particle size fractions delivered from the MDI device will ideally have an MMAD of <10 μm, more preferably between 1-5 μm, and most preferably between 1-3 μm. Examples of formulations and devices for MDIs are provided for in WO 97/47286, WO 98/13031, U.S. Pat. No. 5725841, WO 98/34596, WO 99/36334, WO 98/35888, WO 98/30262.

### Experimental

The following formulations have been tested in pigs:

| | | | | excipients/ | | |
|---|---|---|---|---|---|---|
| Test | | | Tonicity | co- | preserv- | Acyl- |
| nr. | Buffer system | pH | system | solvents | ative | GLP-1 |
| 1 | 8 mM Na₂HPO₄: | 7.4 | manni- | — | phenol | 5 mg/mL |
| | NaH₂PO₄ | | tol | | | |
| 2 | 8 mM Na₂HPO₄: | 7.4 | NaCl | 10:1 m.r. | — | 5 mg/mL |
| | NaH₂PO₄ | | | DPPC | | |

approx. 125 μg/Kg was administered via nebulization using a Maxin® MA3 jet nebulizer (Clinova Medical AB, Malmö, Se) adjusted for an air flow pressure of 5 bar.

Pigs (Landrace×Yorkshire×Duroc, mean weight ca. 20 kg) were anesthetized, intubated, and ventilated. The nebulizer was attached in line on the inspiratory side of the ventilation circuit using a T-piece.

MMAD of aerosol particles were between 4.3-4.8 μm.

Plasma GLP-1 levels were assessed using a validated immunoassay.

The results showed that the GLP-1 compound whereto is attached a lipophilic substituent optionally via a spacer (e.g. Arg³⁴Lys²⁶(N^{ε}-(γ-glutamyl(N^{α}-hexadecanoyl)))-GLP-1(7-37)-OH, referred to as Acyl-GLP-1) was absorbed in vivo via pulmonary delivery.

We claim:

1. A pulmonary liquid or dry formulation comprising a GLP-1 compound whereto is attached a lipophilic substituent optionally via a spacer.

2. The pulmonary formulation of claim 1 wherein said GLP-1 compound is exendin or an analog thereof or a GLP-1 analogue.

3. The pulmonary formulation of claim 2 wherein said GLP-1 compound is exendin-3, exendin-4 or Arg³⁴-GLP-1(7-37)-OH.

4. The pulmonary formulation of any one of claims 1-3 wherein said lipophilic substituent comprises 4-40 carbon atoms.

5. The pulmonary formulation of any one of claims 1-4 wherein said lipophilic substituent is hexadecanoyl.

6. The pulmonary formulation of any one of claims 1-5 wherein a spacer is present.

7. The pulmonary formulation of claim 6 wherein said spacer is γ-Glu or β-Ala.

8. The pulmonary formulation of claim 1 wherein said GLP-1 compound whereto is attached a lipophilic substituent via a spacer is Arg³⁴Lys²⁶ (N^{ε}-(γ-glutamyl(N^{α}-hexadecanoyl)))-GLP-1(7-37)-OH, Arg¹⁸, Leu²⁰, Gln³⁴, Lys³³ (N^{ε}-(γ-aminobutyroyl(N^{α}-hexadecanoyl))) Exendin-4-(7-45)-NH₂ or Arg³³, Leu²⁰, Gln³⁴, Lys¹⁸ (N^{ε}-(γ-aminobutyroyl(N^{α}-hexadecanoyl))) Exendin-4-(7-45)-NH₂.

9. A pulmonary delivery device comprising a formulation according to any one of claims 1-8.

10. A pulmonary delivery device comprising a GLP-1 compound whereto is attached a lipophilic substituent optionally via a spacer.

11. A method for preparing a pulmonary liquid or dry formulation for use in a pulmonary device, said formulation comprising a GLP-1 compound whereto is attached a lipophilic substituent optionally via a spacer.

12. A method for preparing a pulmonary delivery device, said device comprising a GLP-1 compound whereto is attached a lipophilic substituent optionally via a spacer.

13. A method of reducing blood glucose levels, treating diabetes type I, diabetes type II, or obesity, or inhibiting gastric acid secretion, or inhibiting apoptosis of β-cells, comprising administering to a patient in need thereof an effective amount of a GLP-1 compound whereto is attached a lipophilic substituent optionally via a spacer by inhalation so as to deposit said GLP-1 compound whereto is attached a lipophilic substituent optionally via a spacer in the lungs of the patient.

14. Use of a GLP-1 compound whereto is attached a lipophilic substituent optionally via a spacer for the preparation of a pulmonary delivery device for reducing blood glucose levels, treating diabetes type I, diabetes type II, obesity, gastric ulcers, or for inhibition of apoptosis of β-cells.

15. The use according to claim 14, wherein said GLP-1 compound whereto is attached a lipophilic substituent optionally via a spacer is Arg³⁴Lys²⁶(N^{ε}-(γ-glutamyl(N^{α}-hexadecanoyl)))-GLP-1(7-37)-OH.
